SEQUENCE LISTING (19 sequences)

SEQ ID NO: 1 - DNA, 28 bp, Artificial Sequence
  misc_feature: Description of Artificial Sequence MBU-O-37 hIL5Ralpha nt.251-26
gctggtaccatgatcatcgtggcgcatg

SEQ ID NO: 2 - DNA, 21 bp, Artificial Sequence
  misc_feature: Description of Artificial Sequence MBU-O-38 hIL5Ralpha nt.1272-1 25
ctctctcaagggcttgtgttc

SEQ ID NO: 3 - DNA, 30 bp, Artificial Sequence
  misc_feature: Description of Artificial Sequence MBU-O-39 hbetac nt.29-49
gctggtaccatggtgctggcccaggggctg

SEQ ID NO: 4 - DNA, 21 bp, Artificial Sequence
  misc_feature: Description of Artificial Sequence MBU-O-40 hbetac nt.1343-1322
cgactcggtgtcccaggagcg

SEQ ID NO: 5 - DNA, 20 bp, Artificial Sequence
  misc_feature: Description of Artificial Sequence MBU-O-41 hIFNaR1 nt.1384-1403
aaaatttggcttatagttgg

SEQ ID NO: 6 - DNA, 31 bp, Artificial Sequence
  misc_feature: MBU-O-42 hIFNaR1 nt.1743-1764
cgtctcgaggttcatttctggtcatacaaag

SEQ ID NO: 7 - DNA, 20 bp, Artificial Sequence
  misc_feature: Description of Artificial Sequence MBU-O-43 hIFNaR2-1 nt.793-812
aaaataggaggaataattac

SEQ ID NO: 8 - DNA, 33 bp, Artificial Sequence
  misc_feature: Description of Artificial Sequence MBU-O-44 hIFNaR2-1 nt.1210-12 3
cgtctcgagacataataaaacttaatcactggg

SEQ ID NO: 9 - DNA, 28 bp, Artificial Sequence
  misc_feature: Description of Artificial Sequence MBU-O-45 hIFNaR2-2 nt.1626-16 0
cgtctcgagatagttttggagtcatctc

SEQ ID NO: 10 - DNA, 46 bp, Artificial Sequence
  misc_feature: Description of Artificial Sequence MBU-O-278 PacI mutagenesis in IL-5Ralpha/IFNaR2-
cacaagcccttgagagagttaattaaaataggaggaataattactg

SEQ ID NO: 11 - DNA, 46 bp, Artificial Sequence
  misc_feature: Description of Artificial Sequence MBU-O-279 PacI mutagenesis in IL-5Ralpha/IFNaR2-
cagtaattattcctcctattttaattaactctctcaagggcttgtg

SEQ ID NO: 12 - DNA, 43 bp, Artificial Sequence
  misc_feature: Description of Artificial Sequence MBU-O-280 PacI mutagenesis in beta/IFNaR
cctgggacaccgagtcgttaattaaaatttggcttatagttgg

SEQ ID NO: 13 - DNA, 43 bp, Artificial Sequence
  misc_feature: Description of Artificial Sequence MBU-O-281 PacI mutagenesis in beta/IFNaR
ccaactataagccaaattttaattaacgactcggtgtcccagg

SEQ ID NO: 14 - DNA, 30 bp, Artificial Sequence
  misc_feature: Description of Artificial Sequence MBU-O-167 hEPO-R primer nt.10
cggggtaccatggaccacctcggggcgtcc

SEQ ID NO: 15 - DNA, 32 bp, Artificial Sequence
  misc_feature: Description of Artificial Sequence MBU-O-308 hEPO-R primer nt.87
cccttaattaagtccaggtcgctaggcgtcag

SEQ ID NO: 16 - DNA, 49 bp, Artificial Sequence
  misc_feature: Description of Artificial Sequence MBU-O-187 Linker for pMET7-MC
tcgactcagatcttcgatatctcggtaacctcaccggttcctcgagtct

SEQ ID NO: 17 - DNA, 49 bp, Artificial Sequence
  misc_feature: Description of Artificial Sequence MBU-O-188 Linker for pMET7-MC
ctagagactcgaggaaccggtgaggttaccgagatatcgaagatctgag

SEQ ID NO: 18 - DNA, 42 bp, Artificial Sequence
  misc_feature: Description of Artificial Sequence forward primer
ggaattcgccaggcgccaccatgggggtgcacgaatgtcctg

SEQ ID NO: 19 - DNA, 30 bp, Artificial Sequence
  misc_feature: Description of Artificial Sequence reverse primer
gcctcgagtcatctgtcccctctcctgcag

(19) United States
(12) Patent Application Publication
(10) Pub. No.: US 2001/0023062 A1
(43) Pub. Date: Sep. 20, 2001

(54) Eukaryotic cell-based gene interaction cloning

(76) Inventors: Xaveer Van Ostade, Lokeren (BE); Joel S. Vandekerckhove, Loppem (BE); Annick Verhee, Lichtervelde (BE); Jan Tavernier, Balegem (BE)

Correspondence Address: Allen C. Turner, TRASK BRITT, P.O. BOX 2550, Salt Lake City, UT, 84110, US

(21) Appl. No.: 09/771,425

(22) Filed: Jan. 26, 2001

Related U.S. Application Data
(63) Continuation of application No. PCT/EP99/05491, filed on Jul. 27, 1999.

(30) Foreign Application Priority Data
Jul. 28, 1998 (EP) 98202528.0

(51) Int. Cl. A61K 48/00; C12Q 1/00; C12N 5/06

(52) U.S. Cl. 435/4; 435/325; 424/93.21

(57) ABSTRACT

A method for screening compounds for their ability to bind a receptor and/or the screening of compounds that antagonize the binding of a ligand to a receptor. The invention provides an easy and powerful screening method in eukaryotic cells (other than yeast cells), such as insect cells, plant cells or mammalian cells, for ligands of orphan receptors, preferably of the multimerizing receptor type, for unknown ligands of known receptors, preferably multimerizing receptors, and for the genes encoding these ligands.

### CROSS-REFERENCE TO RELATED APPLICATIONS

This application is a continuation of International Application PCT/EP99/05491, filed on Jul. 27, 1999, designating the United States of America, the entire contents of which are incorporated herein by this reference.

### TECHNICAL FIELD

The present invention relates to a method for screening compounds for their ability to bind a receptor and/or the screening of compounds that antagonize the binding of a ligand to a receptor.

### BACKGROUND

Receptors are defined as proteinaceous macromolecules that are often located on cell membranes and that perform a signal transducing function. Many receptors are located on the outer cell membrane. Several receptors possess three domains, the extracellular domain, the transmembrane domain and the cytoplasmic domain. The extracellular domain is capable of specifically binding to a compound, normally called a “ligand”. Signal transduction appears to occur in a variety of ways upon ligand binding, such as, for example, by a conformational change in the structure of the receptor by clustering two or more identical or related receptor-type molecules.

Many receptors have been identified, and the scientific literature has variously divided them into groups, superfamilies, families and/or classes of receptors based on common features, such as tissue distribution of the receptors, nucleic acid or amino acid homology of the receptors, mechanisms of signaling by the receptors or the type of ligand that binds to the receptors. A uniform system of classifying or grouping receptors, however, has not been used in the literature.

It is well established that polypeptide hormones elicit their biological effect by binding to receptors expressed on the surface of responsive cells. At least four families of polypeptide hormone receptors can be defined on the basis of similarity in primary sequence, predicted secondary and tertiary structure and biochemical function. These are 1) the haemopoietin/interferon receptor family; 2) the receptor kinase family; 3) the tumor necrosis factor (TNF)/nerve growth factor (NGF) family; and 4) the family of G-protein coupled receptors. The haemopoietin/interferon family receptors have no intrinsic enzymatic activity; they can be recognized on the basis of their “cytokine receptor homology” (CRH) region in their extracellular domains. This CRH region contains two conserved cysteine bridges and a tryptophan-serin-X-tryptophan-serine motif. The receptor kinase family is characterized by a conserved catalytic kinase domain in the cytoplasmic part of the receptor; the family is subdivided in tyrosine kinase and serine/threonine kinase receptors, on the basis of their substrate specificity. The defining features of members of the TNF/NGF receptor family are located in the extracellular domain and center on a domain that contains 6 cysteine residues. While receptors in the haemopoietin, TNF/NGF and kinase families contain a single transmembrane domain, G-protein coupled receptors traverse the membrane several times. With the exception of the G-protein coupled receptors, cytokine driven multimerization of the receptor subunits appears to be the initial event in signal transduction. While homo- or heterodimerization and trimerization are central to the function of haemopoietin/interferon receptors and TNF/NGF receptors, homodimerization appears a preferred way of receptor kinase action.

A special case is that of the receptor-like protein tyrosine phosphatases. All members possess an intracellular part containing one or two homologous protein tyrosine phosphatase domains, a single membrane spanning region and variable extracellular segments with potential ligand binding capacity.

As described above, cytokine-driven interaction between receptor subunits appears to be the initial event for haemopoietin/interferon receptors. The recognition of the ligand starts with one receptor subunit; this subunit is often called a-subunit in case of heteromeric receptors. After this initial event, there is an association of one or more additional receptor molecules, which is essential for the initiation of the signal transduction and, as an additional effect, it can lead to an increase in affinity of the ligand binding. Receptor clustering leads to activation of the kinase function. The haemopoietin/interferon receptors which, contrary to the tyrosine kinase receptors do not have an intrinsic kinase activity, use the help of the associated “Janus kinases” (JAKs) to phosphorylate the tyrosine residues. Subsequent targets for the JAKs include the JAK molecules themselves, the cytoplasmic part of the receptor and the “Signal Transducers and Activators of Transcription” proteins (STAT). This pathway is called the “JAK/STAT pathway”. Additional pathways, such as the Ras-Raf-mitogen activated protein kinase pathway may also be activated.

Examples of the haemopoietin/interferon receptors are, amongst others, the interleukin-5 (IL-5) receptor, the erythropoietin receptor and the interferon receptor family. The IL-5 receptor is a heteromer consisting of two subunits. The IL-5 receptor a-chain is ligand specific and has a low to intermediate binding affinity. Association with the IL-5 receptor b-chain, that is common with other receptor complexes such as IL-3, results in a high affinity binding complex. Both receptor subunits are required for signaling. Furthermore, signaling requires the cytoplasmic tails of both receptor subunits.

Interferons are classified into two classes. Type I interferons consist of the IFNa group, IFNb, IFNw and the bovine embryonic form, IFNt. IFNg belongs to the second group (type II interferon). The receptor complex of the type I interferons consists of an IFNaR1 subunit and an IFNaR2 subunit. The latter receptor chain exists in three isoforms, resulting from alternative splicing: IFNaR2-1 and IFNaR2-2 are membrane associated but differ in the length of the cytoplasmic domain, whereas IFNaR2-3 is a soluble form.

A lot of information about the signal transduction process of these receptors has been obtained by genetic complementation studies, using the 2fTGH cell line (Pellegrini et al., 1989; Darnell et al., 1994) and the 6-16 promoter (Porter et al., 1988). The human 2fTGH cell line is hypoxanthine-guanine phosphoribosyl transferase (HGPRT) deficient, but contains the xanthine guanine phosphoribosyl transferase (gpt) gene of E. coli, under the control of the type I IFN inducible 6-16 promoter. In cell lines with a functional interferon type I receptor (IFNaR), the 6-16 promoter becomes induced and the gpt gene is transcribed when IFNa or b is added to the medium. The enzyme produced, xanthine guanine phosphoribosyl transferase (XGPRT), is able to complement the HGPRT deficiency. This allows a positive or a negative selection. Positive selection (growth of XGPRT producing cells) is carried out on hypoxanthine aminopterine thymidine (HAT) medium; negative selection (death of XGPRT producing cells) is carried out on DMEM medium with 6-thioguanine (6-TG).

The study of receptor-ligand interactions has revealed a great deal of information about how cells respond to external stimuli. This knowledge has led to the development of several therapeutically important compounds. However, many molecules that control cell growth and development are not yet discovered, and there exist so-called “orphan receptors”, of which the ligand(s) are unknown.

Several methods have been proposed to screen for ligands of orphan receptors. Kinoshita et al. (1995) developed a functional screen in yeast to identify ligands for receptor tyrosine kinases. This method is hampered by the need to have functional expression of the receptor genes in the yeast host. Another yeast system is described in WO/9813513. This system makes use of chimeric Gα proteins in order to couple a mammalian G-protein-coupled receptor to the yeast G-protein intracellular pathway. Also here, the method is restricted to yeast and is thus hampered by the need for functional expression of the mammalian receptor genes in the yeast host. Furthermore, the method is restricted to G-protein-coupled receptors. U.S. Pat. No. 5,597,693 describes a screening method in mammalian cells that is, however, limited to intracellular receptors of the steroid/thyroid superfamily and can not be used for cytokine receptors. PCT International Publication No. WO 95/21930 describes a screening method for cytokine receptors. In this method, ligands are screened after random mutagenesis of a cell line. Only those ligands can be detected of which the expression can be activated by mutagenesis in the cell type used. Moreover, the isolation of the ligand encoding genes is rather complicated. This is a severe restriction for the usefulness of the screening method. In PCT International Publication No. WO 96/02643, a method is described to screen for ligands of the Denervated Muscle Kinase (DMK) receptor and chimeric variants thereof. However, the applicability of this method is rather limited, and there is no direct, rapid way provided to isolate the genetic material encoding the ligand.

### SUMMARY OF THE INVENTION

The present invention provides an easy and powerful screening method in eukaryotic cells, such as insect cells, plant cells or mammalian cells, with the exclusion of yeast cells, for ligands of orphan receptors, preferentially of the multimerizing receptor type, for unknown ligands of known receptors, preferentially multimeric or multimerizing receptors and for the genes encoding these ligands. Hereto, chimeric receptors are constructed, comprising an extracellular domain derived from one protein, preferentially the extracellular domain of a receptor, and a cytoplasmic part derived from another protein which should be a receptor; at least one chimeric receptor is expressed in a eukaryotic host cell which is not a yeast cell. The same eukaryotic host cell comprises a recombinant gene, encoding for a compound of which the expression creates an autocrinic loop, and a reporter system that is activated upon the creation of the autocrinic loop. Preferentially, the compound of which the expression creates an autocrinic loop is a ligand for the chimeric receptor. When this autocrinic loop is closed, the reporter system is switched on, preferentially by the use of a promoter that can be activated as a result of binding the ligand to the chimeric receptor.

All three elements (a first recombinant gene encoding a chimeric receptor, a second recombinant gene encoding the compound, and the reporter system) can be either stably transformed into the eukaryotic cell, or transiently expressed. Transfection methods described in the art can be used to obtain expressed cell. Other examples include non-limiting methods such as calcium-phosphate transfection (Graham and Van der Eb, 1973), lipofection (Loeffner and Behr, 1993) and retroviral gene transfer (Kitamura et al., 1995). Simultaneous expression of several different cDNA products by one cell, which may result in a decreased expression of the relevant cDNA, is ordinarily avoided. Generally, the retroviral gene transfer is preferred since, depending on the virus/cell ratio, an average infection of one virus per cell is obtained.

Moreover, it is clear to people skilled in the art that the autocrinic loop can be more complex and may consist of more than one loop. As a non-limiting example, the recombinant gene may express the ligand of a first (chimeric or non-chimeric) receptor that activates a second gene, which upon activation expresses the ligand of a second receptor, of which the ligand binding results in the induction of the reporter system. It is even not essential that the first and the second receptor are situated within the same cell: it is clear to people skilled in the art that one can work with two cell populations, the first one carrying a recombinant gene, expressing a ligand for a receptor for the second cell, which upon binding of the ligand starts to produce the ligand of the chimeric receptor, situated on the first cell. Binding of the latter ligand to the chimeric receptor then results in the expression of the reporter system.

In a first embodiment, the gpt selection system can be applied to the screening and/or selection of orphan receptors. Hereto, the extracellular domain of the receptor that is studied is fused to the intracellular domain(s) of IFNaR. The receptor studied may be an orphan receptor or a receptor from which not all the ligands are known. The use of the IFN receptor cytoplasmic tails is sufficient for signal transduction, which is required for reporter activation, independent of the function (which may be unknown) of the receptor studied. The ligand is supplied by the creation of an autocrinic loop: cells are transfected by a DNA expression library, where genes, encoding for possible ligands for the orphan receptor, are placed preferentially after a strong, constitutive promoter. It is known, however, to people skilled in the art, that other promoters can be used, such as inducible promoters and even an IFN inducible promoter. The production of the cognate ligand induces the transcription of the gpt gene, enabling a positive selection in HAT medium.

Alternatively, candidate ligands can be added to the medium; survival of the cells in the HAT medium will only be detected when a ligand can activate the orphan receptor.

In a second embodiment, secreted alkaline phosphatase (SEAP) may be used as the reporter system. Cells expressing the reporter system can be identified by measuring the SEAP activity using CSPD (disodium 3-(4-methoxyspirol-1,2-dioxetane-3,2′-(5′-chloro)trichloro {3.3.1.1(3,7)}decan-4-yl)phenyl phosphate) as luminogenic substrate. The invention is not limited to the use of the cytoplasmic tails of the interferon receptor and the gpt selection system, but other receptor systems and/or other inducible promoters and/or other reporter systems and/or other cell lines, known to people skilled in the art, may be used. As a non-limiting example, PC12 cells (Greene et al., 1976), with a chimeric receptor based on the leptin receptor (Tartaglia et al., 1995) and the inducible promoter from the Pancreatitis associated protein I gene may be used. The reporter system may be based upon the detection of the gene product of an inducible gene, as is the case for Green Fluorescent Protein (GFP), a non limiting example, or may be based on modification of a protein already present in the cell (proteolytic cleavage, phosphorylation, complex formation, etc.), such as the systems described by Mitra et al. (1995), Miyawaki et al. (1997) and Romoser et al. (1997). Moreover, optimal reporter activation may require a co-stimulus, as is the case for the leptin-forskolin system.

A further aspect of the invention is the screening of compounds that are antagonists of the ligand-receptor binding. Due to the fact that these compounds can be screened for the toxicity of gpt expression in D-MEM+6-TG medium, it is possible to set up an antagonistic screening system for compounds that inhibit and/or compete with the binding of the ligand to the chimeric receptor. This can be realized by using the autocrinic loop and adding possible inhibitors to the medium, but it is clear to people skilled in the art that, alternatively, the cell can be transformed with genes encoding candidate inhibitors. Expression of an inhibitor would create an anti-autocrinic loop. In this case, the ligand is produced either by an autocrinic loop or is added to the medium, or the receptor may be mutated and/or genetically modified to a form that constitutively initiates the signaling pathway. Such a screening may be useful in the identification of compounds with potential pharmaceutical applications.

A further aspect of the invention is the screening of compounds in the signaling pathway: a host cell, carrying the chimeric receptor and the gene for its ligand, placed after a promoter, in principle inducible by the chimeric receptor, but where the host cell is missing one or more compounds of the signaling pathway, can be transfected by an expression library in order to complement the signaling pathway. Complemented cells will be detected by the activation of the reporter system. This method could be extremely useful in case a receptor with unknown signaling pathway is placed in the autocrinic loop, or before or after the loop that is activating the chimeric receptor.

Still another aspect of the invention is the screening of compounds that are involved in the secretory pathway: as the ligand for the chimeric receptor needs to be secreted in order to activate the receptor, both compounds that block the secretion, or compounds that can complement a mutation in the secretory pathway can be screened.

### BRIEF DESCRIPTION OF THE SEVERAL VIEWS OF THE DRAWINGS

FIG. 1: Transient co-transfection of pSV-SPORT-IL-5Rα/IFNaR2-2, pSV-SPORT-βc/IFNaR1 and p6-16SEAP in 2ftGH cells and analysis of induction of SEAP activity. 24 hours after transfection, cells were left unstimulated or were stimulated with IFNO (positive control) or IL-5 (1 and 2 ng/ml). Samples from the medium were taken 24 hours after stimulation, and SEAP activity was measured using CSPD as a luminogenic substrate (PHOSPHA-LIGHT™ Kit, Tropix). The amount of light produced was determined in a Topcount luminometer (Packard).

FIG. 2: Transient transfection of pSV-SPORT-EpoR/IFNaR1+pSV-SPORT-EpoR/IFNaR2-2, pSV-SPORT-EpoR/IFNaR1 or pSV-SPORT-EpoR/IFNaR2-2 in 2fTGH 6-16SEAP Clone 5 cells. 24 hours after transfection, cells were left unstimulated or were stimulated with IFNβ (1 ng/ml; positive control) or Epo (5 ng/ml). Samples from the medium were taken 24 hours after stimulation and SEAP activity was measured using CSPD as luminogenic substrate (Phospha-light kit, Tropix). The amount of light was determined in a Topcount luminometer (Packard).

FIG. 3: Survival of 2fTGH IL-5Rα/IFNaR2-2+βc/IFNaR1 clone C cells, transfected with dilutions of the vector pEFBOS-hIL-5syn in irrelevant DNA. Formation of an autocrinic loop results in survival of the cells in HAT medium. Fifteen days after transfection, photographs of representative regions in each Petri dish were taken.

FIG. 4: Induction of SEAP activity in IL-5Rα/IFNaR2-2+βc/IFNaR1 clone E, transfected with dilutions of the vector pMET7-hIL-5syn in irrelevant DNA and co-transfected with the p6-16 plasmid. Formation of an autocrinic loop results in activation of the 6-16 promoter followed by secretion of SEAP. Samples from the medium were taken 24 hours after transfection and SEAP activity was measured using CSPD as luminogenic substrate (Phospha-light kit, Tropix). The amount of light produced was determined in a Topcount luminometer (Packard).

FIG. 5A. Induction of SEAP activity in 2fTGH IL-5Rα/IFNaR2-2+βc/IFNaR1 clone E cells, transfected with dilutions of the vector pMET7-hIL-5syn in an EL4 cDNA library that was expressed in the eukaryotic expression vector pACGGS. All dilutions were co-transfected with the p6-16 plasmid. Negative control was pACGGS-EL4cDNA+p6-16SEAP. Transfection was performed according to the Ca-phosphate method. Formation of an autocrinic loop results in activation of the 6-16 promoter followed by secretion of SEAP. Samples from the medium were taken 24 hours after transfection and SEAP activity was measured using CSPD as luminogenic substrate (phospha-light kit, Tropix). The amount of light produced was determined in a Topcount luminometer (Packard).

FIG. 5B. The same conditions were used as in FIG. 5A with the exception that transfection was performed according to the lipofection method, using Superfect reagent (Qiagen).

FIG. 6: Induction of SEAP activity in 2fTGH 6-16SEAP EpoR/IFNaR2-2 clone 4 cells, transfected with dilutions of the vector pMET7-moEpo in an EL4 cDNA library that was expressed in the eukaryotic expression vector pACGGS. All dilutions were co-transfected with the p6-16 plasmid. Negative control was pACGGS-EL4cDNA+p6-16SEAP. Formation of an autocrinic loop results in activation of the 6-16 promoter followed by secretion of SEAP. Samples from the medium were taken 24 hours after transfection and SEAP activity was measured using CSPD as luminogenic substrate (Phospha-light kit, Tropix). The amount of light produced was determined in a Topcount luminometer.

### DETAILED DESCRIPTION OF THE INVENTION

### Definitions

The following definitions are set forth to illustrate and define the meaning and scope of the various terms used to describe the invention herein.

Multimerizing receptor: every receptor of which the interaction with or binding of the ligand results in the multimerization of receptor components, and/or every protein that can be identified by people skilled in the art as such a receptor on the base of its amino acid sequence and/or protein structure. Interaction is often the binding to the receptor, but can for instance also be binding to one component of a receptor complex, which subsequently associates with other receptor components to form the receptor complex. Another example is the transient interaction of a ligand with a receptor component leading to a conformational change or allowing a specific enzymatic modification leading to signal transduction.

“Multimerization” can be homo- or heterodimerization, homo- or heterotrimerization, and so forth, up to complex formations of multiple proteins.

“Orphan receptor” means every receptor, preferably a multimerizing receptor or protein with known receptor components of which no ligand is known, that is interacting or binding to this receptor and, as a consequence, initiating or inhibiting the signaling pathway.

“Ligand” means every compound that can interact with or bind to a receptor, preferentially a multimerizing receptor and that is initiating or inhibiting the signaling pathway by its interaction with or binding to the receptor.

“Unknown ligand” means every compound that can interact with or bind to a receptor, preferentially a multimerizing receptor and that is initiating or inhibiting the signaling pathway by its interaction with or binding to the receptor, but for which this interaction or binding has not yet been demonstrated.

“Compound” means any chemical or biological compound, including simple or complex inorganic or organic molecules, peptides, peptido-mimetics, proteins, antibodies, carbohydrates, phospholipids, nucleic acids or derivatives thereof.

“Extracellular domain” means the extracellular domain of a receptor and/or orphan receptor, or a functional fragment thereof characterized by the fact that it still can interact with or bind to a known and/or unknown ligand or a fragment thereof fused to other amino acid sequences, characterized by the fact that it still can interact with or bind to a known and/or unknown ligand or a fragment from a non-receptor protein that can interact with or bind to a known and/or unknown ligand.

“Bind” or “binding” means any interaction, be it direct (direct interaction of the compound with the extracellular domain) or indirect (interaction of a compound with one or more identical and/or non-identical compounds resulting in a complex of which one or more compounds can interact with the extracellular domain), that results in initiating or inhibiting the signaling pathway of the chimeric receptor

“Cytoplasmic domain” means the cytoplasmic part of a receptor, or a functional fragment thereof, or a fragment thereof fused to other amino acid sequences, capable of initiating the signaling pathway of the receptor and of inducing a reporter system.

“Chimeric receptor” means a functional receptor comprising an extracellular domain of one receptor and the cytoplasmic domain of another receptor.

“Reporter system” means every compound of which the synthesis and/or modification and/or complex formation can be detected and/or be used in a screening and/or selection system. The reporter system can be, as a non-limiting example, a gene product encoding an enzymatic activity, a colored compound, a surface compound or a fluorescent compound.

“Autocrinic loop” means every succession of events by which a cell carrying a receptor allows the synthesis of a known or unknown compound that, directly or indirectly, induces the activation of the receptor.

“Anti-autocrinic loop” means every succession of events by which a cel, carrying a receptor allows the synthesis of a known or unknown compound that, directly or indirectly, inhibits the binding of a ligand and/or unknown ligand to the receptor.

“Signaling pathway” means every succession of events after the binding of a ligand and/or unknown ligand to an extracellular domain of a natural occurring or chimeric receptor whereby the binding can result in the induction and/or repression of a set of genes.

“Selection” means isolation and/or identification of cells in which the reporter system is activated or isolation and/or identification of cells in which the reporter system is not activated.

The invention is further explained by use of the following illustrative examples:

### EXAMPLE I

### Construction of the Chimeric Receptors

I.1. Construction of IL-5R/IFNaR chimeric receptors

I.1.1 Construction in the pcDNA3 vector

All polymerase chain reactions (PCR) were performed using the Expand High Fidelity PCR system kit (Boehringer Mannheim). This kit is supplied with an enzyme mix containing thermostable Taq DNA and Pwo DNA polymerases (Barnes et al, 1994). The IL-5Rα extracellular domain sequence (amino acids 1-341, not including the last Trp 342 residue) was amplified by PCR using the forward primer MBU-O-37 that contains a Kpn I site and the reverse primer MBU-O-38 (Table 1). The sequence encoding the βc extracellular domain (amino acids 1-438, not including the last Val439 residue) was PCR amplified using the forward primer MBU-O-39 which also contains a KpnI site and the reverse primer MBU-O-40. A forward primer MBU-O-41 was used with a reverse primer MBU-O-42, which contains an XhoI site, to amplify the sequence that codes for the IFNaR1 transmembrane (TM) and intracellular (IC) domain (amino acids 436-557, including the last residue of the extracellular domain, Lys436). The forward primer MBU-O-43 was used to amplify the sequence encoding the IFNaR2-1 transmembrane and intracellular domains (amino acids 243-331, including the last residue of the extracellular domain, Lys243) and the IFNaR2-2 TM and IC domains (amino acids 243-515, including the last residue of the extracellular domain, Lys 243), respectively in combination with the reverse primers MBU-O-44 and MBU-O-45, containing an XhoI site. After gel purification, and phosphorylation, six combinations of PCR fragments encoding for the EC on the one hand and for the TM+IC domains on the other hand, were ligated and subsequently used as input DNA in a second PCR reaction:

1) IL-5Rα EC domain fragment+IFNaR1 IC and TM domain fragments, using MBU-O-37 and MBU-O-42 as forward and reverse primers, respectively.

2) IL-5Rα EC domain fragment+IFNaR2-1 IC and TM domain fragments, using MBU-O-37 and MBU-O-44 as forward and reverse primers, respectively.

3) IL-5Rα EC domain fragment+IFNaR2-2 IC and TM domain fragments, using MBU-O-37 and MBU-O-45 as forward and reverse primers, respectively.

4) βc EC domain fragment+IFNaR1 IC and TM domain fragments, using MBU-O-39 and MBU-O-42 as forward and reverse primers, respectively.

5) βc BC domain fragment+IFNaR2-1 IC and TM domain fragments, using MBU-O-39 and MBU-O-44 as forward and reverse primers, respectively.

6) βc EC domain fragment+IFNaR2-2 IC and TM domain fragments, using MBU-O-39 and MBU-O-45 as forward and reverse primers, respectively.

The resultant blunt PCR fragments, coding for the hybrid receptors, were isolated by agarose gel electrophoresis, digested with KpnI-XhoI and ligated into the KpnI-XhoI opened pcDNA3 vector (Invitrogen).

The constructs were checked by DNA sequence analysis and named as follows: pcDNA3-IL-5Rα/IFNaR1, pcDNA3-IL-5Rα/IFNaR2-1, pcDNA3-IL-5Rα/IFNaR2-2, pcDNA3-βc/IFNaR1, pcDNA3-βc/IFNaR2-1 and pcDNA3-βc/IFNaR2-2.

TABLE 1
oligonucleotides used for construction of chimeric receptors and IL-5
expression vectors.

| | | forward/ | |
|---|---|---|---|
| Number | Specification | reverse | Sequence (5′-3′) |
| MBU-O-37 | hIL5Ralpha nt.251-268 | Forward | GCTGGTACCATGATCATCGTGGCGCATG (SEQ.ID.NO. 1) |
| MBU-O-38 | hIL5Ralpha nt.1272-1252 | Reverse | CTCTCTCAAGGGCTTGTGTTC (SEQ.ID.NO. 2) |
| MBU-O-39 | hbetac nt.29-49 | Forward | GCTGGTACCATGGTGCTGGCCCAGGGGCTG |
| | | | (SEQ.ID.NO. 3) |
| MBU-O-40 | hbetac nt.1343-1322 | Reverse | CGACTCGGTGTCCCAGGAGCG (SEQ.ID.NO. 4) |
| MBU-O-41 | hIFNaR1 nt.1384-1403 | Forward | AAAATTTGGCTTATAGTTGG (SEQ.ID.NO. 5) |
| MBU-O-42 | hIFNaR1 nt.1743-1764 | Reverse | CGTCTCGAGGTTCATTTCTGGTCATACAAAG |
| | | | (SEQ.ID.NO. 6) |
| MBU-O-43 | hIFNaR2-1 nt.793-812 | Forward | AAAATAGGAGGAATAATTAC (SEQ.ID.NO. 7) |
| MBU-O-44 | hIFNaR2-1 nt.1210-1234 | Reverse | CGTCTCGAGACATAATAAAACTTAATCACTGGG |
| | | | (SEQ.ID.NO. 8) |
| MBU-O-45 | hIFNaR2-2 nt.1626-1608 | Reverse | CGTCTCGAGATAGTTTTGGAGTCATCTC (SEQ.ID.NO. 9) |
| MBU-O-278 | PacI mutagenesis in IL- | Forward | CACAAGCCCTTGAGAGAGTTAATTAAAATAGGAGGA |
| | 5Ralpha/IFNaR2-2 | | ATAATTACTG (SEQ.ID.NO. 10) |
| MBU-O-279 | PacI mutagenesis in IL- | Reverse | CAGTAATTATTCCTCCTATTTTAATTAACTCTCTCAAG |
| | 5Ralpha/IFNaR2-2 | | GGCTTGTG (SEQ.ID.NO. 11) |
| MBU-O-280 | PacI mutagenenesis in | Forward | CCTGGGACACCGAGTCGTTAATTAAAATTTGGCTTAT |
| | beta/IFNaR1 | | AGTTGG (SEQ.ID.NO. 12) |
| MBU-O-281 | PacI mutagenenesis in | Reverse | CCAACTATAAGCCAAATTTTAATTAACGACTCGGTGT |
| | beta/IFNaR1 | | CCCAGG (SEQ.ID.NO. 13) |
| MBU-O-167 | hEPO-R primer nt. 105 | Forward | CGGGGTACCATGGACCACCTCGGGGCGTCC |
| | | | (SEQ.ID.NO. 14) |
| MBU-O-308 | hEPO-R primer nt. 872 | Reverse | CCCTTAATTAAGTCCAGGTCGCTAGGCGTCAG |
| | | | (SEQ.ID.NO. 15) |
| MBU-O-187 | Linker for pMET7-MCS | Sense | TCGACTCAGATCTTCGATATCTCGGTAACCTCACCGG |
| | | | TTCCTCGAGTCT (SEQ.ID.NO. 16) |
| MBU-O-188 | Linker for pMET7-MCS | Antisense | CTAGAGACTCGAGGAACCGGTGAGGTTACCGAGATA |
| | | | TCGAAGATCTGAG (SEQ.ID.NO. 17) |

I.1.2. Construction in the pSV-SPORT vector and insertion of a PacI site

Alternatively, chimeric receptors were tested in the pSV-SPORT expression vector (Life Technologies). This vector contains an SV40 early promoter that is normally weaker than the CMV promoter of the pcDNA3 plasmid.

The genes for the chimeric receptors in pcDNA3-IL-5Rα/IFNaR2-2 and pcDNA3-βc/IFNaR1 were isolated by Asp728 and XhoI digestion and agarose gel electrophoresis, followed by insertion in the Asp718-SalI opened pSV-SPORT vector. The resulting constructs were verified by sequence analysis and named pSV-SPORT-IL-5Rα/IFNaR2-2 and pSV-SPORT-βc/IFNaR1.

In addition, we inserted a unique Pac restriction site immediately preceding the last amino acid codon of each extracellular domain (Trp341 and Val438 for IL-5Rα and βc, respectively). This enabled us to quickly exchange the IL-5R extracellular domains with the extracellular domains of other receptors. Insertion mutagenesis was performed with the QuickChange site-directed mutagenesis kit (Stratagene), using the oligonucleotides MBU-O-278 (sense) and MBU-O-279 (antisense) for IL-5Rα/IFNaR2-2 and MBU-O-280 (sense) and MBU-O-281 (antisense) for βc/IFNaR1 (table 1). As a result, two amino acids (Leu-Ile) were inserted in the membrane-proximal region of the extracellular domain, which did not interfere with receptor functionality. The resulting plasmids were named pSV-SPORT-IL5RαP/IFNaR2-2 and pSV-SPORT-βcP/IFNaR1

I.2. Construction of EPO-R/IFNaR chimeric receptors

RNA was prepared from 5×10⁶ TF-1 cells according to the procedure of the RNeasy kit (Qiagen), and dissolved in 50 μl water from which 10 μl was used for RT-PCR. To these, 2 μl (2 μg) of oligodT (12-18 mer; Pharmacia) was added and incubated at 70° C. for 10 min. After chilling on ice for 1 min., cDNA was prepared by adding 4 μl of RT buffer (10×; Life Sciences), 1 μl dNTP's (20 mM; Pharmacia), 2 μl DTT (0.1M) and 1 μl of MMLV reverse transcriptase (200U; superscript; Life Technologies) so that the total volume was 20 μl. Incubations were successively at RT for 10 min., 42° C. for 50 min., 90° C. for 5 min. and 0° C. for 10 min.. Following this, 0.5 μl RnaseH (2 U; Life Technologies) was added and the mixture was incubated at 37° C. for 20 min., followed by chilling on ice. For PCR amplification of the DNA, 5 μl of this mixture was diluted in 17 μl water followed by addition of 1 μl dNTP's (20 mM), 5 μl Pfu buffer (10×; Stratagene), and 10 μl (100 ng) of forward and reverse primer for EPO-R (MBU-O-167 and MBU-O-308, respectively, see table 1). The PCR was started at 94° C. for 2 min. during which 2 μl Pfu enzyme (5 U; Stratagene) was added (hot start) and followed by 40 cycles with denaturation at 92° C. (1 min.), hybridization between 55 till 59° C. (1 min.; with an increasing temperature gradient over 4° C. during the 40 cycles) and polymerization at 72° C. (3 min.; with an increasing time elongation of 0.05 min. during every cycle, but only in the last 25 cycles). To finalize, the reaction was hold on 72° C. for 12 min. and chilled to 4° C. A band of correct size was isolated from an agarose gel and the DNA was digested with PacI and KpnI and inserted into the PacI-KpnI opened pSV-SPORT-IL-5RαP/IFNaR2-2 or pSV-SPORT-βcP/IFNaR1 vectors. The resultant vectors were named pSV-SPORT-EPO-R/IFNaR2-2 and EPO-R/IFNaR1, respectively.

### EXAMPLE II

### Functionality of the Chimeric Receptors

II.1. IL-5 can activate the 6-16 promoter via IL-5R/IFNaR chimeric receptors.

II.1.1. Activation of 6-16 gpt allows selection of stable colonies.

The following nine combinations of plasmids were transfected in 2fTGH cells:

1. pcDNA3-IL-5Rα/IFNaR1+pcDNA3-βc/IFNaR1

2. pcDNA3-IL-5Rα/IFNaR1+pcDNA3-βc/IFNaR2-1

3. pcDNA3-IL-5Rα/IFNaR1+pcDNA3-βc/IFNaR2-2

4. pcDNA3-IL-5Rα/IFNaR2-1+pcDNA3-βc/IFNaR1

5. pcDNA3-IL-5Rα/IFNaR2-1+pcDNA3-βc/IFNaR2-1

6. pcDNA3-IL-5Rα/IFNaR2-1+pcDNA3-βc/IFNaR2-2

7. pcDNA3-IL-5Rα/IFNaR2-2+pcDNA3-βc/IFNaR1

8. pcDNA3-IL-5Rα/IFNaR2-2+pcDNA3-βc/IFNaR2-1

9. pcDNA3-IL-5Rα/IFNaR2-2+pcDNA3-βc/IFNaR2-2

pcDNA3 alone was used for mock transfection.

Transfection was performed according to the calcium phosphate method (Graham and van der Eb (1973)). For each plasmid, 10 μg DNA was used (20 μg of pcDNA3 for mock transfection). The precipitate was made up in 1 ml and left on the cells overnight (5×10⁵ cells/transfection/Petri dish). The dishes were then washed twice with Dulbecco's PBS (Life Technologies) and cells were left in DMEM (Life Technologies). 48 hours later, DMEM medium +G418 (Calbiochem; 400 μg/ml) was added. 3 days later, cells from every transfection were trypsinized with 5 ml 0.05% trypsin/0.02% EDTA solution (Life Technologies) and seeded in three wells of a 6-well microtiterplate. The day after, 1) HAT medium (Life Technologies) alone+G418, 2) HAT medium+G418+500 U/ml IFNα2b (PeproTech, Inc) or 3) HAT medium+G418+1 ng/ml IL-5 (produced in Sf9 cells using published methodologies) was added. 6 days later, small colonies appeared only in the IL-5Rα/IFNaR1+βc/IFNaR2-2 and IL-5Rα/IFNaR2-2+βc/IFNaR1 transfections, when the cells were incubated with HAT+G418+IL-5, indicating that these IL-5R/IFNaR chimeric receptors were functional in that they transmitted the signal to activate the 6-16 promoter. Growth in the HAT medium alone resulted in clear colony formation. Incubation with 500 U/ml IFNα resulted in only 50-100 colonies (see, Table 2).

TABLE 2

| | HAT | HAT + IL-5 | HAT + IFNα |
|---|---|---|---|
| IL-5Rα/IFNaR1 + βc/IFNaR1 | — | — | +/−75 |
| IL-5Rα/IFNaR1 + βc/IFNaR2-1 | — | — | +/−50 |
| IL-5Rα/IFNaR1 + βc/IFNaR2-2 | — | 3 | +/−50 |
| IL-5Rα/IFNaR2-1 + βc/IFNaR1 | — | — | +/−75 |
| IL-5Rα/IFNaR2-1 + βc/IFNaR2-1 | — | — | +/−100 |
| IL-5Rα/IFNaR2-1 + βc/IFNaR2-2 | — | — | +/−100 |
| IL-5Rα/IFNaR2-2 + βc/IFNaR1 | — | 13 | +/−100 |
| IL-5Rα/IFNaR2-2 + βc/IFNaR2-1 | — | — | +/−100 |
| IL-5Rα/IFNaR2-2 + βc/IFNaR2-2 | — | — | +/−50 |
| mock | — | — | +/−100 |

The experiment was repeated twice, with slight modifications in the procedures according to time of adding supplements, changing media and length of incubation times. However, similar results were obtained.

To isolate single clones, cells stabile transfected with the combinations pcDNA3-IL-5Rα/IFNaR1+pcDNA3-βc/IFNaR2-2 or pcDNA3-IL-5Rα/IFNaR2-2 +pcDNA3-βc/IFNaR1, were further cultivated for two days in DMEM medium+HT supplement, allowing cells to switch back to normal DMEM medium. Single cells were isolated by limited dilution in a 96-well microtiterplate and resulting colonies were further grown in DMEM for two weeks for depletion of gpt, and stored. 6 colonies of each transfection were further investigated on their IL-5 responsiveness by re-analyzing their growth behaviour in HAT medium alone, HAT medium+IL-5, or DMEM medium.

Using an inverted microscope, cell survival was visually followed during a two week period and selection of an optimal clone was based on 1) rapid growth in HAT+IL-5 which correlates with rapid growth in DMEM, and 2) pronounced cell death in HAT alone. One clone was selected for each combination: IL-5Rα/IFNaR1+βc/IFNaR2-2 clone B and IL-5Rα/IFNaR2-2+βc/IFNaR1 clone C.

2ftGH cells that were stabile transfected with the pSV-SPORT IL-5Rα/IFNaR2-2+pSV-SPORT βc/IFNaR1 vectors were isolated essentially the same way with the exception that selection in G418 medium was omitted. For each plasmid, 10 μg DNA was used (20 μg of pSV-SPORT for mock transfection). The precipitate was made up in 1 ml and left on the cells overnight (5×10⁵ cells/transfection/Petri dish). The dishes were then washed twice with Dulbecco's PBS and cells were left in DMEM. 24 hours later, cells from every transfection were trypsinized with 5 ml 0.05% trypsin/0.02% EDTA solution (Life technologies) and seeded in three wells of a 6-well microtiterplate. The day after, 500 U/ml IFNα or 1 ng/ml IL-5 was added or cells were left unstimulated and 24 hours later the medium was removed and replaced by HAT medium with the same stimuli or without stimulus. About 14 days later, small colonies appeared, when the cells were incubated with HAT+IL-5. Growth in HAT medium alone resulted in clear colony formation, while in all transfections, incubation with 500 U/ml IFNα resulted in a confluent monolayer. Isolation of single colonies was performed essentially the same way as described above. The degree of responsiveness of single colonies to IL-5 was determined by investigating growth in HAT medium supplemented with IL-5, versus cell death in HAT medium alone. Alternatively, cell growth in medium containing 6-thioguanine (6-TG) versus cell death in 6-TG containing medium supplemented with IL-5, was also determined. The survival or death was determined visually during a two-week period, using an inverted microscope A clone with the best response to IL-5 was called 2fTGH IL-5Rα/R2-2+βc/R1 CloneE.

The cells developed at this stage could already serve as an assay system for the evaluation of exogeneously added ligands.

II.1.2. Construction of p6-16SEAP and development of the 2fTGH-6-16SEAP stabile cell line

Although formation of stable colonies is a reliable and reproducible assay to investigate chimeric receptor activation, this method suffers from the disadvantage that it is very time-consuming and cannot be used for quantification of receptor functionality. We therefore constructed a plasmid wherein the 6-16 promoter was cloned into the pSEAP vector (Tropix), upstream the reporter gene coding for secreted alkaline phosphatase (SEAP). A HindIII fragment that contained the entire 6-16 promoter was isolated from the plasmid 6-16luci (gift from Sandra Pellegrini, Institut Pasteur, Paris) and inserted in the HindIII-opened pSEAP vector so that the 6-16 promoter was in front of the SEAP gene. The resultant plasmid was named p6-16SEAP.

Stabile 6-16SEAP transfected 2fTGH cell lines were obtained by co-transfection of 20 μg p6-16SEAP with 2 μg pBSpac/deltap (obtained from the Belgian Coordinated Collections of Microorganisms, BCCM) in the 2fTGH cells. The latter plasmid contained a gene for puromycin resistence under control of the constitutive SV40 early promoter. Selection on puromycin was on the basis of methods described in the art. We chose 3 μg puromycin/ml as an optimal concentration for selection of puromycin-resistant 2ftGH cells. Single colonies were isolated by limited dilution in 96-well microtiterplates and investigated on SEAP production after treatment with IFNa or b versus no stimulus. The clones 2fTGH-6-16SEAPclone2 and 2ftGH-6-16SEAPclone5 were selected, based on an optimal stimulation window.

II.1.3. Activation of the 6-16SEAP reporter by IL-5 in transient transfection assays

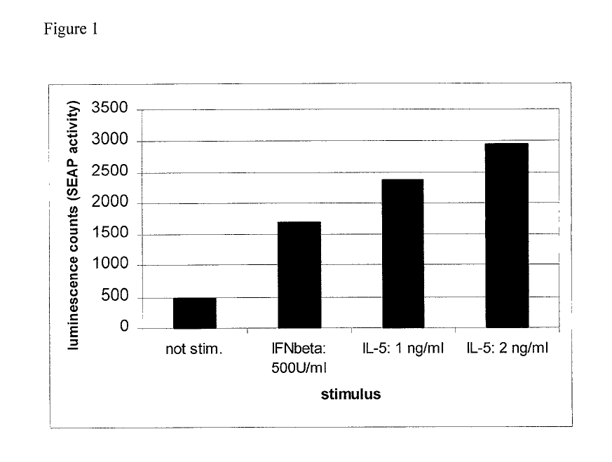

10 μg of pSV-SPORT-IL-5Rα/IFNaR2-2 and 10 μg of pSV-SPORT-βc/IFNaR1 were co-transfected in 2ftGH cells, together with 10 μg of the plasmid p6-16SEAP. Transfection was performed according to the Ca-phosphate procedure (Graham and Van der Eb, 1973). The precipitate was made up in 1 ml and equally dispersed over four wells in a 6-well microtiterplate (165 μl/10⁵ cells/well) and left on the cells overnight. Cells were washed twice the next day (2× with Dulbecco's PBS)and further grown in DMEM medium for 24 hours. The day after, no stimulus, IFNα (500U/ml; IFNb1a, gift from P. Hochman, Biogen, Cambridge, UK) or IL-5 (1 and 2 ng/ml) was added and the cells were left for another 24 hours. Finally, samples of medium from each well were taken to assay for SEAP activity with the Phospha-Light kit (Tropix), using CSPD as a luminogenic substrate and light production was measured in a Topcount luminometer (Canberra-Packard). Comparison with untreated cells shows a 2.5-fold increase in SEAP activity when the cells were treated with IFNβ as compared to untreated cells, and a 5- or 6-fold increase when cells were stimulated with 1 or 2 ng/ml IL-5, respectively (FIG. 1).

II.2. Erythropoietin can activate the 6-16 promoter via Epo-R/IFNaR chimeric receptors

II.2.1. Activation of 6-16 SEAP in transient transfection assays

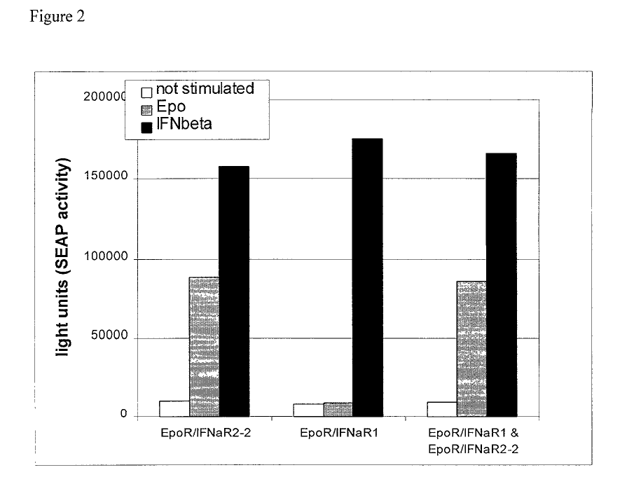

20 μg of pSV-SPORT-EPO-R/IFNaR2-2 alone, 20 μg of pSV-SPORT-EPO-R/IFNaR1 alone, 10 μg of pSV-SPORT-EPO-R/IFNaR1+10 μg of pSV-SPORT-EPO-R/IFNaR2-2 or 20 μg of pUC18 alone (mock; Pharmacia) were transfected in 2ftGH-6-16SEAP clone2 cells, using the Ca-phosphate method (Graham and Van der Eb, 1973). The precipitate was made up in 1 ml and left on the cells for six hours (5×10⁵ cells/transfection/Petri dish). The dishes were then washed twice with Dulbecco's PBS and cells were further grown in DMEM. After 24 hours, cells from every transfection were trypsinized with 5 ml 0.05% trypsin/0.02% EDTA solution (Life Technologies) and seeded in three wells of a 6-well microtiterplate. The next day, no stimulus, IFNα (500U/ml) or erythropoietin (EPO, 0.5 U/ml, R&D systems) was added and the cells were left for another 24 hours. Finally, samples of medium from each well were taken to assay for SEAP activity with the Phospha-Light kit (Tropix), using CSPD as a luminogenic substrate and light production was measured in a Topcount luminometer. Comparison with untreated cells shows a 4-fold increase in SEAP activity when the cells were treated with IFNβ or IFNα. There was no induction of SEAP by EPO in the cells transfected with the EPO-R/IFNaR1 chimer alone. However, a 8 to 9-fold induction of SEAP activity by EPO was observed in those cells transfected with the EPO-R/IFNaR1 +EPO-R/IFNaR2-2 constructs or with the EPO-R/IFNaR2-2 construct alone (FIG. 2), indicating that at least EPO-R/IFNaR2-2 can be activated by EPO and transmits a signal resulting in 6-16 promoter activation.

II.2.2. Development of 2fTGH cells, stabile expressing the EpoR/IFNaR2-2 chimeras

2fTGH-6-16SEAP clone5 cells were transfected with 20 μg of pSV-SPORT-EpoR/R2-2 and 2 μg pcDNA1 /Neo. A calcium phosphate precipitate was made up in 1 ml according to the method of Graham and Van der Eb (1973), and left on the cells overnight (8×10⁵ cells/transfection/Petri dish). The dishes were then washed twice with PBS and cells were left in DMEM. 48 hours later, DMEM medium+G418 (400 μg/ml) was added and refreshed every 3-4 days for a period up to 14 days. Individual cells were isolated by limited dilution in a 96-well microtiterplate. The degree of responsiveness of single colonies to Epo was determined by investigating growth in HAT medium supplemented with Epo, versus cell death in HAT medium alone. Alternatively, cell growth in medium containing 6-thioguanine (6-TG) versus cell death in 6-TG containing medium supplemented with Epo, was also determined. The survival or death was determined visually during a two-week period, using an inverted microscope. Furthermore, the 2fTGH 6-16SEAP clone 5 cells have the 6-16SEAP construct stabile transfected, allowing fast determination of Epo responsiveness by measurement of SEAP induction. On the basis of these assays, 2fTGH-6-16SEAP EpoR/2-2 clone 4 showed the highest responsiveness for Epo and was selected for further analysis.

### EXAMPLE III

### Activation of the Chimeric Receptors Upon Endogenously Produced Ligand

III.1. Construction of the vectors pEFBos-hIL-5syn and pMET7-hIL-5syn for constitutive eukaryotic expression of IL-5

The gene for hIL-5syn was isolated from the pGEM1-hIL-5syn vector (Tavernier et al. 1989) by Sal I digestion and agarose gelelectrophoresis. The fragment was cloned into the Sal I opened pEFBOS vector (gift from Nagata, S., Osaha Bioscience Institute, Japan). As a result, the hIL-5syn gene was cloned downstream the promoter for human elongation factor 1a (HEF1a, Mizushima et al., 1990) and the resultant plasmid was named pEFBos-hIL-5syn. In addition, the Sal I fragment was also cloned into the pMET7MCS vector. This vector was constructed by replacing the DNA encoding the leptin receptor long form (Lrlo) in the plasmid pMET7-Lrlo (gift from L. Tartaglia, Millennium, Cambridge), with the DNA coding for a multicloning site (Sal I-Bgl II-EcoR V-BstE II-Age I-Xho I-Xba I), formed by hybridization of the oligonucleotides MBU-O-187 and MBU-O-188 (table 1). Here, the hIL-5syn gene was cloned downstream the hybrid SRα promoter (Takebe et al. 1988) and the plasmid was named pMET7-hIL-5syn.

III.2. Construction of pMET7-moEpo for constitutive eukaryotic expression of monkey Epo

The plasmid pMFEpo2 (gift from Dr. C. Laker, Heinrich-Pette-institut), was used as input DNA for PCR amplification of monkey Epo cDNA, using a forward primer (GGAATTCGCCAGGCGCCACCATGGGGGTGCACGAATGTCCTG) (SEQ. ID. NO. 18) that contains a kozak sequence and an EcoR1 site and a reverse primer (GCCTCGAGTCATCTGTCCCCTCTCCTGCAG) (SEQ. ID. NO. 19), containing a XhoI site. The PCR was performed with Pfu polymerase (Stratagene) and the obtained product of ±±600 bp was purified by gel extraction and digested with EcoRI-XhoI. This fragment was inserted into the pMET7mβc/SEAP vector. This plasmid encodes for a chimeric protein (alkaline phosphatase fused to the C-terminal end of the mouse IL-5 beta common (mβc) chain), downstream the SRα promoter. The mβc/SEAP gene was removed by an EcoRI-XhoI digest, allowing ligation of the moEpo fragment into the opened pMET7 vector. The resulting plasmid was named pMET7-moEpo.

III.3. Chimeric receptors allow survival selection upon endogenously produced ligand

The plasmids pEFBOS-hIL-5syn or the pUC18 vector (mock) were used for transfection of 2ftGH cells that stabile expressed the IL-5Rα/IFNaR2-2+βc/IFNaR1 chimeras (2ftGH clone C cells). Transfection was performed overnight according to the Ca-phosphate method (Graham and Van der Eb, 1973). The precipitates were made up in 1 ml and left on the cells overnight (5×10⁵ cells/transfection/Petri dish). The next day, cells were washed twice with Dulbecco's PBS. Two days later, cells were incubated on HAT medium alone, after which cell survival was visually followed using an inverted microscope. Three days later, a clear difference in cell confluency between pEFBOS-hIL-5syn and mock transfected cells was visible. Cells, transfected with pEFBOS-hIL-5syn, were trypsinised and a limited dilution was set up in a 96-well microtiterplate. Six colonies surviving in HAT medium without IL-5 supplementation could be isolated, indicating that these cells produced IL-5 and stimulated the chimeric receptor in an autocrinic fashion.

III.4. Determination of the minimum amount of pEFBOS-hIL-5syn DNA required for generation of an IL-5 autocrinic loop

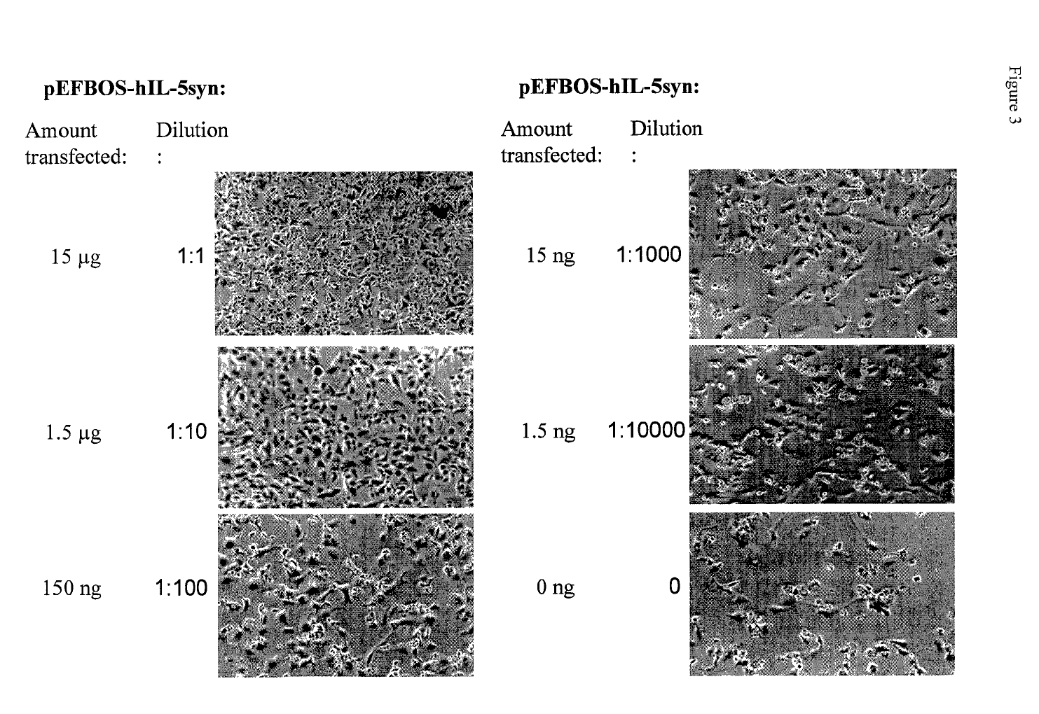

The occurrence of a relevant cDNA in a pool of irrelevant cDNA within a cDNA library was mimicked by making serial dilutions of the expression vectors containing the gene for hIL-5 in irrelevant vector. A 1:10 dilution series of pEFBOS-hIL-5syn DNA in irrelevant DNA (pcDNA.3) was set up: 1.5 (1/10), 0.15 (1/100), 0.015 (1/1000) and 0.0015 (1/10000) μg of pEFBOS-hIL-5syn DNA were added to 15 μg pcDNA3 DNA and transfected in the IL-5Rα/IFNaR2-2+βc/IFNaR1 clone C cells. Positive and negative controls were 15 μg of pEFBOS-hIL-5syn and 15 μg of pcDNA3, respectively. Transfection was according to the Ca-phosphate procedure (Graham and Van der Eb, 1973). The precipitates were made up in 1 ml and left on the cells overnight (5×10⁵ cells/transfection/Petri dish). Following washing (2× with Dulbecco's PBS), DMEM medium was added for 24 hours after which it was changed to HAT medium. Cells were visually followed using an inverted microscope and 15 days after transfection, photographs of representative regions in every petri dish were taken. All of the petri dishes, containing cells transfected with one of the pEFBOS-hIL-5syn dilutions, showed a marked increase in cell number as compared to the negative control (FIG. 3). Hence, transfection of as little as 1.5 ng pEFBOS-hIL-5syn in 15 μg total DNA (1:10⁴ dilution) is sufficient to generate an autocrine loop that allows cell survival in HAT medium.

III.5. Determination of the minimum amount of pMET7-hIL-5syn DNA required for generation of an IL-5 autocrinic loop

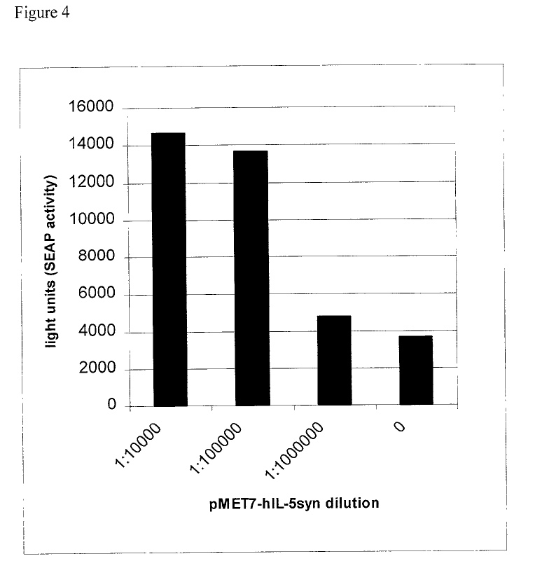

A dilution series of pMET7-hIL-5syn DNA in irrelevant DNA (pCDNA3) was set up containing: 4 ng (1/10⁴), 400 pg (1/10⁵), and 40 pg (1/10⁶) of pMET7-hIL-5syn DNA that were added to 40 μg pCDNA3 DNA and transfected in the 2fTGH IL-5Rα/IFNaR2-2+bc/IFNaR1 Clone E cells (stabile transfected with pSV-SPORT-IL-5Rα/IFNaR2-2+pSV-SPORT-βc/IFNaR1). As a negative control, 40 μg of pCDNA3 alone was used. 10 mg p6-16 SEAP was added to all samples. Every precipitate was prepared in 1 ml according to the Ca-phosphate procedure (Graham and Van der Eb, 1973), from which 165 μl (6.8 mg of total DNA) was brought onto 10⁵ cells in the well of a 6-well microtiterplate. The precipitate was left on the cells overnight after which cells were washed twice with Dulbecco's PBS. Cells were further grown in DMEM medium. After 24 hours, medium samples were taken from each well and SEAP activity was measured using the Phospha-Light assay (Tropix). Luminescence was measured in a Topcount luminometer. Transfection of the cells with 68 pg pMET7-hIL-5syn in 6.8 μg total DNA (1/10⁵ dilution of pMET7-hIL-5syn DNA), still resulted in a clear SEAP production, as compared to the negative control, indicating that an autocrine loop was formed (FIG. 4).

III.6. Determination of the minimum amount of pMET7-hIL-5syn DNA required for generation of an IL-5 autocrinic loop by dilution in the pACGGS-EL4cDNA library

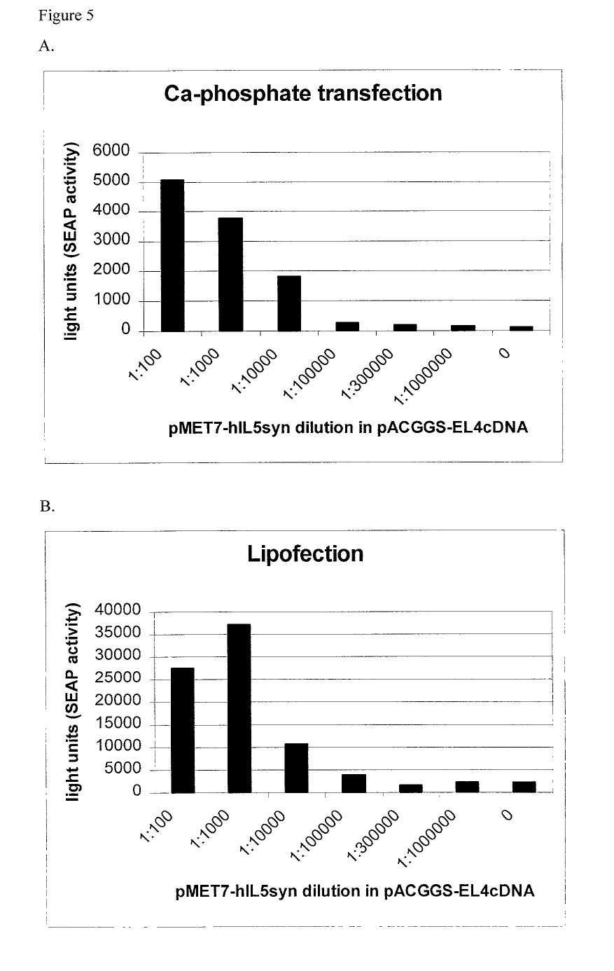

To optimally mimic the occurrence of the cDNA coding for the relevant ligand in a large pool of irrelevant cDNAs, we diluted the pMET7-hIL-5syn plasmid in a cDNA library. This library was made from the mouse EL4 lymphoma cell line and cDNAs were inserted into the vector pACGGS under control of the chicken b-actin promoter. 125 ng (1/10²), 12.5 ng (1/10³), 1.25 ng (1/10⁴), 125 pg (1/10⁵)), 42 pg (⅓×10⁵) and 12.5 μg (1/10⁶) of pMET7-moEpo DNA were added to 9.4 μg pACGGS-EL4cDNA and 3.1 μg p6-16SEAP. As a negative control, we transfected 9.4 μg of pACGGS-EL4cDNA+3.1 μg of p6-16SEAP. Every precipitate was prepared in 500 μl, according to the Ca-phosphate procedure (Graham and Van der Eb, 1973), and 165 μl (±4 μg total DNA) was brought onto 10⁵ 2fTGH 6-16SEAP EpoR/IFNaR2-2 Clone 4 cells in the well of a 6-well microtiterplate. The precipitate was left on the cells for 6 hours after which the cells were washed twice with Dulbecco's PBS. Cells were further grown in DMEM medium. After 18 hours, medium samples were taken from each well and SEAP activity was measured using the Phospha-Light assay (Tropix). Luminescence was measured in a Topcount luminometer. Transfection of the cells with 400 pg pMET7-hIL-5syn in 4 μg total DNA (1/10⁴ dilution), still resulted in a clear SEAP production, as compared to the negative control, indicating that an autocrine loop was formed (FIG. 5a).

The same dilutions were set up for transfection according to the lipofection method (Loeffner and Behr, 1993). Here, a total of 2 μg was transfected into the cells (4×10⁵ cells/well), in combination with 2.5 μl of DNA carrier (Superfect; Qiagen). Transfection was performed according to the manufacturers guidelines. The mixture was left on the cells for 2 hours after which the cells were washed. After 18 hours, medium samples were taken from each well and SEAP activity was measured as described above. Also here, transfection of the cells with 200 pg pMET7-hIL-5syn in 2 μg total DNA (1/10⁴ dilution), still resulted in a clear SEAP production, as compared to the negative control, indicating that an autocrine loop was formed (FIG. 5b).

III.7. Determination of the minimum amount of pMET7-moEpo DNA required for generation of an Epo autocrinic loop by dilution in the pACGGS-EL4cDNA library

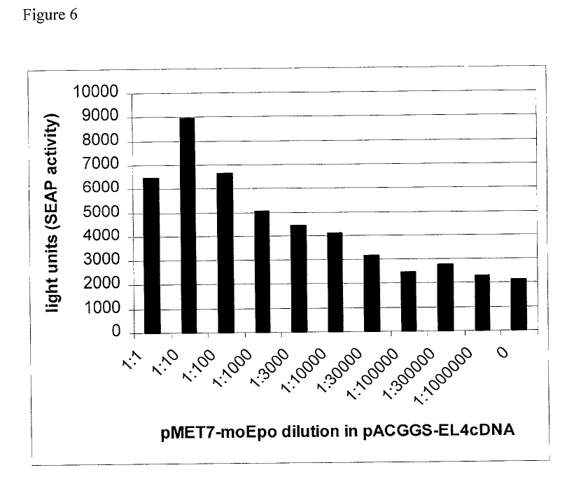

To optimally mimic the occurrence of the cDNA coding for the relevant ligand in a large pool of irrelevant cDNAs, we diluted the pMET7-moEpo plasmid in a cDNA library. This library was made from the mouse EL4 lymphoma cell line and cDNAs were inserted into the vector pACGGS under control of the chicken β-actin promoter. 1.25 μg (1/10), 125 ng (1/10²), 12.5 ng (1/10³), 4.2 ng (⅓×10³), 1.25 ng (1/10⁴), 420 μg (1/3×10⁴), 125 μg (1/10⁵), 42 pg (⅓×10⁵) and 12.5 pg (1/10⁶) of pMET7-moEpo DNA were added to 9.4 μg pACGGS-EL4cDNA and 3.1 μg p6-16SEAP and transfected in the 2fTGH 6-16SEAP EpoR/IFNaR2-2 Clone 4 cells. Although in principle the addition of p6-16 SEAP is not required because of the stable integration of p6-1 6SEAP in these cells, the addition of p6-16 SEAP to the transfection mixture increased the sensitivity of this assay. Negative and positive controls were 9.4 μg of pACGGS-EL4cDNA+3.1 μg of p6-16SEAP, and 9.4 μg pMET7-moEpo+3.1 μg of p6-16SEAP, respectively. Every precipitate was prepared in 500 μl, according to the Ca-phosphate procedure (Graham and Van der Eb, 1973), and 165 μl (about 4 mg total DNA) was brought onto 10⁵ cells in the well of a 6-well microtiterplate. The precipitate was left on the cells for 6 hours after which cells were washed twice with Dulbecco's PBS. Cells were further grown in DMEM medium. After 18 hours, medium samples were taken from each well and SEAP activity was measured using the Phospha-Light assay (Tropix). Luminescence was measured in a Topcount luminometer. Transfection of the cells with 400 pg pMET7-hIL-5syn in 4 μg total DNA (1/10⁴ dilution), still resulted in a clear SEAP production, as compared to the negative control, indicating that an autocrine loop was formed (FIG. 6).

### REFERENCES

Barnes, W. M. (1994) PCR amplification of up to 35-kb DNA with high fidelity and high yield from lambda bacteriophage templates. Proc. Nat. Acad. Sci. USA, 91, 2216-2220.

Darnell, J. E., Kerr, I. M. and Stark, G. R. (1994) Jak-STAT pathways and transcriptional activation in response to IFNs and other extracellular signaling proteins. Science, 264, 1415-1421.

Graham, F. L. and Van der Eb (1973) A new technique for the assay of infectivity of human adenovirus 5 DNA. Virology, 52, 456-467.

Greene, L. A. and Tischler, A. (1996) Establishment of a noradrenergic clonal line of rat adrenal pheochromocytoma cells which respond to nerve growth factor. Proc. Natl. Acad. Sci. USA, 73, 2424-2428.

Kinoshita, N., Minshull, J. And Kirschner, M. W. (1995): The identification of two novel ligands of the FGF receptor by a yeast screening method and their activity in Xenopus development. Cell, 83, 621-630.

Kitamura, T., Onishi, M., Kinoshita, S., Shibuya, A., Miyajima, A. and Nolan, G. P. (1995) Efficient screening of retroviral cDNA expression libraries. Proc. Natl. Acad. Sci. USA 92, 9146-9150.

Loeffner, J.-P. and Behr, J.-P. (1993) Gene transfer into primary and established mammalian cell lines with lipopolyamine-coated DNA. Meth. Enzymology 217, 599-618.

Mitra, R. D., Silva, C. M. and Youvan, D. C. (1996) Fluorescence resonance energy transfer between blue-emitting and red-shifted excitation derivatives of the green fluorescent protein. Gene, 173, 13-17.

Miyawaki, A., et al (1997): Fluorescent indicators for Ca²⁺ based on green fluorescent proteins and calmodulin. Nature, 388,882-887.

Mizushima, S. And Nagata, S. (1990) pEFBOS, a powerful mammalian expression vector. Nucleic acids Res. 18, 5322.

Pellegrini, S., John, J., Shearer, M., Kerr, I. M. and Stark, G. R. (1989) Use of a selectable marker regulated by alpha interferon to obtain mutations in the signaling pathway. Mol. Cell. Biol., 9, 4605-4612.

Pereschini, A., Lynch, J. A. and Rosomer, V. A. (1997) Novel fluorescent indicator proteins for monitoring intracellular Ca²⁺. Cell Calcium, 22, 209-216.

Porter, A. C. G., Chemajovsky, Y., Dale, T. C., Gilbert, C. S., Stark, G. R. and Kerr, I. M. (1988) Interferon response element of the human gene 6-16. EMBO, 7, 85-92.

Romoser, V. A., Hinkle, P. M. and Persechini, A. (1997) Detection in living cells of Ca²⁺ dependent changes in the fluorescence emission of an indicator composed of two green fluorescent protein variants linked by a calmodulin-binding sequence. A new class of fluorescent indicators. J. Biol. Chem., 16, 13270-13274.

Takebe, Y., Seiki, M., Fujisawa, J., Hoy, P., Yokota, K., Arai, K., Yoshida, M. And Arai, N. (1988) SR alpha promoter: an efficient and versatile mammalian cDNA expression system composed of the simian virus 40 early promoter and the R-U5 segment of human T-cell leukemia virus type I long terminal repeat. Mol. Cell. Biol., 8, 466-472.

Tartaglia, L. A., Dembski, M., Weng, X., Deng, N., Culpepper, J., Devos, R., Richards, G. J., Camfield, L. A., Clark, F. T. and Deeds, J. (1994) Identification and expression cloning of a leptin receptor, OB-R. Cell, 83, 1263-1271.

Tavernier, J., Devos, R., Van der Heyden, J., Hauquir, G., Bauden, R., Fache, I., Kawashima, E., Vandekerckhoven J., Contreras, R. and Fiers, W. (1989) Expression of human and murine interleukin-5 in eukaryotic systems. DNA, 8, 491-501.

What is claimed is:

1. A eukaryotic cell comprising:
a first recombinant gene encoding a chimeric receptor;
a second recombinant gene encoding a compound the expression of which creates an autocrinic or anti-autocrinic loop; and
a reporter system that is activated or inactivated upon the creation of said autocrinic or anti-autocrinic loop.

2. The eukaryotic cell of claim 1 wherein the cell is any eukaryotic cell other than yeast.

3. The eukaryotic cell of claim 1 or 2 wherein the chimeric receptor is a multimeric or multimerizing receptor.

4. The eukaryotic cell of claim 1, claim 2, or claim 3, wherein said second recombinant gene is functionally incorporated after a constitutive promoter.

5. The eukaryotic cell of any one of claims 1 through 4 wherein said reporter system is activated as a result of a ligand binding to said chimeric receptor.

6. The eukaryotic cell of claim 1 wherein a cytoplasmic part of the chimeric receptor is a cytoplasmic part of one of at least one interferon receptor subunit.

7. The eukaryotic cell of claim 1 wherein the reporter system comprises E. coli xanthine-guanine phosphoribosyl transferase (gpt).

8. The eukaryotic cell of claim 6 wherein said reporter system is placed under control of a 6-16 promoter.

9. The eukaryotic cell of claim 4 wherein said second recombinant gene is inserted after an SRa or HEF1a promoter.

10. The eukaryotic cell of claim 1 wherein the cell is a 2fTGH cell.

11. A method of screening for compounds that interfere with the binding of a ligand with the extracellular part of a chimeric receptor and/or with the signaling pathway of the cytoplasmic part of a chimeric receptor, the method comprising:
providing the eukaryotic cell of any one of claims 1 through 10;
reacting a series of compounds with said eukaryotic cell; and
determining the activity of each element of said series of compounds.

12. A method of screening for orphan receptors and for unknown ligands, said method comprising:
transforming an eukaryotic host cell with a gene encoding a chimeric receptor;
transforming said eukaryotic host cell with a gene encoding a reporter system inducible by a ligand's binding to said chimeric receptor;
transforming said host cell with a gene encoding for a ligand of said chimeric receptor; and
selecting for cells in which the reporter system is activated or inactivated.

13. A method for screening compounds that interfere with the binding of a ligand to a receptor and/or with the signaling pathway of a receptor, said method comprising:
transforming a eukaryotic host cell with a gene encoding a chimeric receptor;
transforming said host cell with a reporter system inducible by a ligand's binding to said chimeric receptor;
transforming said host cell with a gene encoding an inhibitor of the ligand binding to said chimeric receptor;
transforming said host cell with a gene encoding a ligand for said chimeric receptor and/or supplying said ligand to the host cell; and
selecting for cells in which the reporter system is activated or inactivated.

14. A kit, comprising a eukaryotic host cell and one or more transformation vectors, which upon the transfection of said cell with said vector or vectors, results in the eukaryotic cell of any one of claim 1 through 10.

15. A method of screening for orphan receptors and unknown ligands comprising:
providing a eukaryotic cell comprising:
a first recombinant gene encoding a chimeric receptor;
a second recombinant gene encoding a compound, the expression of which creates an autocrinic or anti-autocrinic loop;
a reporter system that is activated or inactivated upon the creation of said autocrinic or anti-autocrinic loop;
reacting a series of compounds with said eukaryotic cell;
assaying the activity of each element of said series of compounds; and
based on said assaying, determining the presence or absence of orphan receptors and unknown ligands.

16. The method according to claim 15 wherein said series of compounds comprise genes encoding candidate inhibitors.

17. The method according to claim 16 wherein said inhibitors create an autocrinic or anti-autocrinic loop.

18. The method according to claim 15 wherein said unknown ligands are produced by an autocrinic or anti-autocrinic loop.

19. The method according to claims 15 wherein said orphan receptors may be mutated and/or genetically modified to a form that constitutively initiates the signaling pathway.

20. A pharmaceutical composition comprising the eukaryotic cell of claim 1.
